(19) United States
(12) Patent Application Publication
(10) Pub. No.: US 2007/0005101 A1
(43) Pub. Date: Jan. 4, 2007

(54) Filter catheter

(75) Inventors: Gary Fahey, Rosscahill County (IE); Ronald Kelly, Athlone (IE); Patrick Griffin, Castlegar (IE); Eamon Brady, Elphin (IE)

Correspondence Address: RISSMAN JOBSE HENDRICKS & OLIVERIO, LLP, ONE STATE STREET, SUITE 800, BOSTON, MA, 02109, US

(73) Assignee: Salviac Limited, Dublin (IE)

(21) Appl. No.: 11/406,819

(22) Filed: Apr. 18, 2006

Related U.S. Application Data
(60) Provisional application No. 60/672,431, filed on Apr. 18, 2005.

(51) Int. Cl. A61M 29/00

(52) U.S. Cl. 606/200

(57) ABSTRACT

A filter retrieval catheter for retrieving an embolic protection filter (50) from a vasculature. The catheter comprises a catheter shaft (2) having a rapid exchange guidewire opening (4) in a sidewall of the catheter shaft (2) to facilitate rapid exchange of the catheter over a guidewire (5). To prevent buckling or kinking of the catheter shaft (2) in the region of the guidewire opening (4) during retrieval of the embolic protection filer (50), the catheter comprises two elongate reinforcement elements (6, 3) extending along the catheter shaft (2). The first reinforcement element (6) comprises an elongate wire element extending from a proximal end located proximally of the guidewire opening (4) to a distal end located distally of the guidewire opening (4). The second reinforcement element (3) comprises an elongate tubular element extending from a proximal end located adjacent to and distally of the guidewire opening (4) to a distal end (9) located further distally of the guidewire opening (4). The tube (3) tapers distally radially outwardly adjacent to the distal end (9). This tapered part (9) of the tube (3) is fixedly attached to the interior surface of the catheter shaft (2).

### RELATED APPLICATION

This application claims the benefit of U.S. Patent Application Ser. No. 60/672,431, filed Apr. 18, 2005, the entire teachings of which are incorporated herein by reference.

### INTRODUCTION

This invention relates to a filter catheter. In particular this invention relates to a filter catheter suitable for rapid exchange over a guidewire.

During advancement of a retrieval catheter through a potentially narrow and/or tortuous vasculature, there is a risk of damage being caused to the vasculature. In addition, a retrieval catheter may be subject to relatively large compressive forces during retrieval of an object, such as an embolic protection filter with a large embolic load, into the catheter. A risk of buckling or kinking of a retrieval catheter may therefore arise during retrieval.

This invention is aimed at providing a filter catheter which addresses at least some of these difficulties.

### STATEMENTS OF INVENTION

According to the invention there is provided a filter catheter comprising:—
a catheter shaft having a lumen extending at least partially therethrough;
the catheter shaft having a rapid exchange guidewire opening in a wall of the catheter shaft, the guidewire opening being located a substantial distance distally of a proximal end of the catheter for rapid exchange of the catheter over a guidewire; and
at least one reinforcement extending along the catheter shaft within the catheter shaft lumen to reinforce the catheter shaft in the region of the guidewire opening;
the reinforcement comprising a tubular element having a lumen extending therethrough aligned with the guidewire opening.

The tubular reinforcement acts to facilitate push to be transmitted distally without adversely affecting the radial flexibility of the catheter.

In one embodiment of the invention the tubular element extends distally of the guidewire opening. A proximal end of the tubular element may be located adjacent to the guidewire opening and a distal end of the tubular element may be located distally of the guidewire opening. In one case the tubular element terminates proximally of a distal end of the catheter. A distal end of the tubular element may be attached to the catheter shaft. The distal end of the tubular element may be attached to an internal surface of the catheter shaft. At least part of the tubular element may taper distally radially outwardly.

In one embodiment the longitudinal axis of the tubular element is radially aligned with the longitudinal axis of the guidewire opening. The catheter shaft lumen may comprise a guidewire lumen through which a guidewire may be passed for rapid exchange of the catheter over the guidewire. The tubular element lumen may comprise a guidewire lumen through which a guidewire may be passed for rapid exchange of the catheter over the guidewire.

According to the invention there is provided a filter catheter comprising:—
a catheter shaft;
the catheter shaft having a rapid exchange guidewire opening in a wall of the catheter shaft, the guidewire opening being located a substantial distance distally of a proximal end of the catheter for rapid exchange of the catheter over a guidewire; and
at least one reinforcement extending along the catheter shaft to reinforce the catheter shaft in the region of the guidewire opening.

In one embodiment of the invention the catheter shaft has a reception space into which an object may be retrieved. The reinforcement may be substantially elongate. The catheter may comprise a first reinforcement and a second reinforcement.

In one case the catheter shaft proximally of the guidewire opening is relatively stiff to provide the catheter with sufficient column strength and buckle strength during retrieval of an object, such as an embolic protection filter with a large embolic load, into the reception space, and the catheter shaft distally of the guidewire opening is relatively flexible to facilitate advancement of the catheter through a potentially narrow and/or tortuous vasculature without causing damage to the vasculature. The reinforcement acts to reinforce the column strength and buckle strength of the catheter shaft in the region of the guidewire opening to prevent buckling or kinking of the catheter shaft at the transition between the proximal stiff catheter shaft and the distal flexible catheter shaft during retrieval.

In one embodiment the reinforcement extends distally of the guidewire opening. A proximal end of the reinforcement may be located proximally of the guidewire opening and a distal end of the reinforcement may be located distally of the guidewire opening. A proximal end of the reinforcement may be located distally of the guidewire opening and a distal end of the reinforcement may be located further distally of the guidewire opening. The reinforcement may extend distally to a distal end of the catheter. The reinforcement may terminate proximally of a distal end of the catheter.

In another embodiment the catheter shaft has a lumen extending at least partially therethrough, and the reinforcement extends along the catheter shaft within the catheter shaft lumen. By extending the reinforcement within the lumen of the catheter shaft, this arrangement minimises the crossing profile of the retrieval catheter. The catheter shaft lumen may comprise a guidewire lumen through which a guidewire may be passed for rapid exchange of the catheter over the guidewire. The guidewire opening may face in a direction substantially parallel to the longitudinal axis of the catheter shaft. Because the guidewire opening faces parallel to the catheter shaft, the overall crossing profile of the retrieval catheter and the guidewire at the guidewire opening is minimised. The guidewire opening may face in a direction which subtends an acute angle with the longitudinal axis of the catheter shaft. In one case the guidewire opening faces proximally.

In a further embodiment the longitudinal axis of the reinforcement is radially offset from the longitudinal axis of the guidewire opening. The longitudinal axis of the reinforcement may be radially aligned with the longitudinal axis of the guidewire opening. The reinforcement may have a lumen extending therethrough aligned with the guidewire opening. The reinforcement lumen may comprise a guidewire lumen through which a guidewire may be passed for rapid exchange of the catheter over the guidewire. The reinforcement may comprise a guide to guide passage of a guidewire into the reinforcement lumen. The guide may comprise a funnel. In one case the funnel is provided at a distal end of the reinforcement. The reinforcement may comprise a tubular element.

In another embodiment the reinforcement has a small cross sectional area relative to the cross sectional area of the guidewire opening. The reinforcement may comprise a wire element.

In a further embodiment the reinforcement has a cross sectional area substantially equal to the cross sectional area of the guidewire opening.

At least part of the reinforcement may taper distally radially outwardly. At least part of reinforcement may taper distally radially inwardly. The tapering part of the reinforcement may be located adjacent to a distal end of the reinforcement. The tapered part of the reinforcement facilitates ease of attachment of the reinforcement to the catheter shaft.

In one case a distal end of the reinforcement is attached to the catheter shaft. In another case a distal end of the reinforcement is detached from the catheter shaft. A proximal end of the reinforcement may be attached to the catheter shaft.

In one embodiment the stiffness of the reinforcement is greater than the stiffness of the catheter shaft. The second moment of area of the catheter shaft may be greater than the second moment of area of the reinforcement.

The catheter shaft may taper proximally radially inwardly towards the guidewire opening. The catheter may comprise a guide to guide passage of a guidewire through the guidewire opening. The reinforcement may comprise the guide. The guide may be provided by an end face of the reinforcement.

In another embodiment the catheter shaft has a substantially uniform outer diameter from the guidewire opening to a distal end of the catheter shaft. The outer diameter of the catheter shaft may vary along the length of the catheter between the guidewire opening and the distal end of the catheter shaft. The catheter shaft may have a reduced diameter portion. The reduced diameter portion may extend longitudinally for a part of the distance between the guidewire opening and the distal end of the catheter shaft. The reduced diameter portion may extend longitudinally for substantially the full distance between the guidewire opening and the distal end of the catheter shaft.

In one case the invention provides a filter retrieval catheter. In another case the invention provides a filter delivery catheter.

### BRIEF DESCRIPTION OF THE DRAWINGS

The invention will be more clearly understood from the following description of some embodiments thereof, given by way of example only, with reference to the accompanying drawings, in which:—

FIG. 1 is a cross-sectional, side view of a filter catheter according to the invention and a guidewire;

FIG. 2 is an enlarged, cross-sectional, side view of a part of the filter catheter of FIG. 1 and the guidewire;

FIGS. 2(a) and 2(b) are partially cross-sectional, side views of the filter catheter of FIG. 1, in use;

FIGS. 2(c) and 2(d) are views similar to FIGS. 1 and 2 of another filter catheter according to the invention;

FIGS. 3 to 6 are cross-sectional, side views of other filter catheters according to the invention;

FIG. 7 is a cross-sectional, side view of the filter catheter of FIG. 6, in use;

FIGS. 8 to 13 are cross-sectional, side views of other filter catheters according to the invention;

FIGS. 14 and 15 are partially cross-sectional, side views illustrating retrieval of an embolic protection filter into a catheter;

FIGS. 16 and 17 are cross-sectional, side views of further filter catheters according to the invention; and

FIG. 18 is a partially cross-sectional, side view of another filter catheter according to the invention.

### DETAILED DESCRIPTION

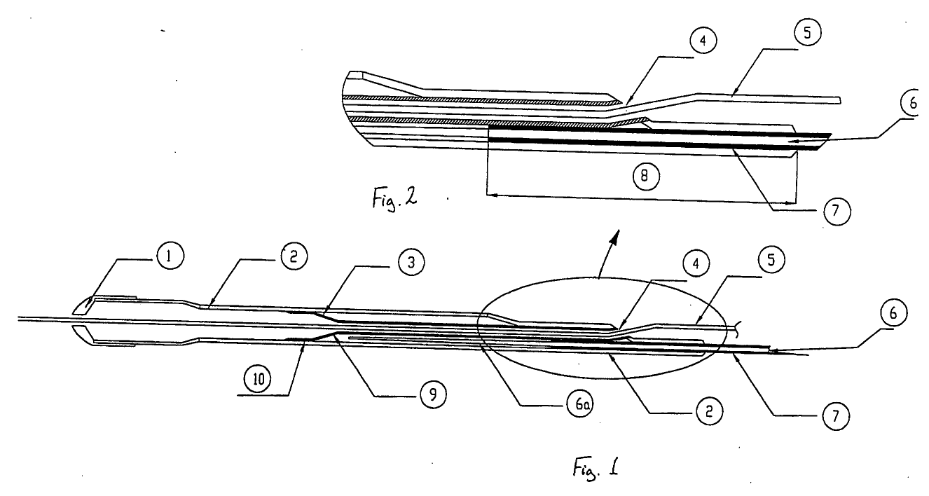

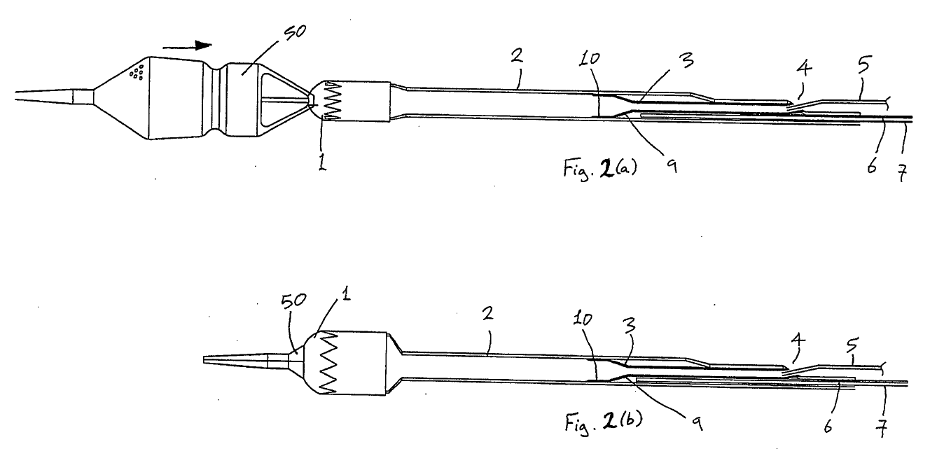

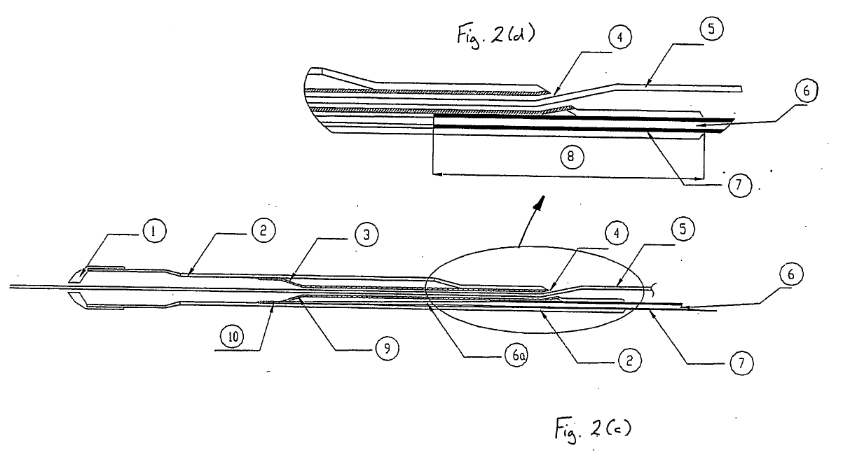

Referring to the drawings, and initially to FIGS. 1 to 2(b) thereof, there is illustrated a filter retrieval catheter according to the invention, which is suitable for retrieving an embolic protection filter 50 from a vasculature. The catheter is particularly suitable for retrieving the type of embolic protection filters described in International patent applications published under numbers WO 01/80777, and WO 03/055412, the relevant contents of which are incorporated herein by reference.

The catheter comprises a catheter shaft 2 having a guidewire opening 4 in a sidewall of the catheter shaft 2. The guidewire opening 4 is a rapid exchange guidewire opening and is located a substantial distance distally of a proximal end of the catheter shaft 2 to facilitate rapid exchange of the catheter over a guidewire 5. In this case, the guidewire opening 4 is located approximately 30 cm from a distal end 1 of the catheter shaft 2.

The guidewire opening 4 faces proximally and is aligned for passage of the guidewire 5 out through the opening 4 in a direction which subtends an acute angle with the longitudinal axis of the catheter shaft 2 (FIG. 2).

Distally of the guidewire opening 4, the catheter shaft 2 has a guidewire lumen extending from the guidewire opening 4 to the distal end 1 of the catheter shaft 2. The catheter shaft 2 has an open mouth at the distal end 1, and the guidewire lumen is in communication with the open mouth and with the guidewire opening 4. In this manner, a guidewire 5 may be passed through the open mouth into the guidewire lumen, through the guidewire lumen and out through the guidewire opening 4 for rapid exchange of the catheter over the guidewire 5.

The guidewire lumen acts as a reception space to enable an embolic protection filter 50 to be retrieved from a vasculature into the guidewire lumen through the open mouth.

Proximally of the guidewire opening 4, the catheter shaft 2 is of a relatively stiff material to ensure that the catheter has sufficient column strength and buckle strength to enable an embolic protection filter 50 to be retrieved into the catheter. Retrieval of the embolic protection filter 50 may involve relatively large forces being exerted on the catheter shaft 2 as the filter 50 with a potentially large embolic load is collapsed and retrieved through the open mouth and into the guidewire lumen (FIGS. 2(a) and 2(b)).

The distal end 1 of the catheter shaft 2 preferably has an expansile tip to accommodate retrieval of a potentially large embolic filter into the catheter shaft 2, as illustrated in FIG. 2(b).

Distally of the guidewire opening 4, the catheter shaft 2 is of a relatively flexible material to ensure that the catheter is sufficiently trackable and flexible to prevent damage being caused during advancement of the catheter through a potentially narrow and/or tortuous vasculature.

To prevent buckling or kinking of the catheter shaft 2 in the region of the guidewire opening 4 during retrieval of the embolic protection filter 50, the catheter comprises two elongate reinforcement elements 6, 3 extending along the catheter shaft 2 within the guidewire lumen. The reinforcements 6, 3 have respective stiffnesses greater than the stiffness of the catheter shaft 2, and respective second moments of area less than the second moment of area of the catheter shaft 2. This arrangement results in the reinforcements 6, 3 acting to reinforce the column strength and buckle strength of the catheter shaft 2 in the region of the guidewire opening 4 during retrieval. The reinforcements provide a linear transition in stiffness. The inner tube 3 is made from a higher stiffness (E) polymer to provide additional compression resistance during retrieval. Even though the inner tube 3 has a high material modulus (E), its smaller outer and inner diameters means that the 2^{nd} moment of Area (I) value for the inner tube 3 is lower than for the outer tube 2.

The combined EI value for the outer tube 2—inner tube 3 construction is lower than for a single tube made from the same material.

The first reinforcement element 6 comprises an elongate wire element extending from a proximal end located proximally of the guidewire opening 4 to a distal end located distally of the guidewire opening 4. The wire 6 extends through a wire lumen in the catheter shaft 2 proximally of the guidewire opening 4, exits the wire lumen distally of the guidewire opening 4 at a lumen outlet, and extends through the guidewire lumen to the distal end of the wire 6. The proximal end of the wire 6 is fixedly attached to the interior surface of the catheter shaft 2, for example by means of welding.

As illustrated in FIG. 2, the longitudinal axis of the wire 6 is radially offset from the longitudinal axis of the guidewire opening 4, and the cross sectional area of the wire 6 is small relative to the cross sectional area of the guidewire opening 4.

A polymer jacket 7, for example of polyethylene, is provided around the wire 6 and extending along the wire 6.

At a distal region 6a, the wire 6 tapers distally radially inwardly (FIG. 1). This taper provides a further means of stiffness transition across the guidewire opening 4.

The second reinforcement element 3 comprises an elongate tubular element extending from a proximal end located adjacent to and distally of the guidewire opening 4 to a distal end 9 located further distally of the guidewire opening 4. In this case, the distance between the proximal end and the distal end 9 is approximately 15 cm. The longitudinal axis of the tube 3 is aligned with the longitudinal axis of the guidewire opening 4. As illustrated in FIG. 2, the cross sectional area of the tube 3 is substantially equal to the cross sectional area of the guidewire opening 4.

The tube 3 has a guidewire lumen extending therethrough. The proximal end of the guidewire lumen is aligned with and is in communication with the guidewire opening 4, and the distal end of the guidewire lumen is aligned with and is in communication with the guidewire lumen of the catheter shaft 2. In this manner, a guidewire 5 may be passed from the guidewire lumen of the catheter shaft 2 through the guidewire lumen of the tube 3, and out through the guidewire opening 4 for rapid exchange of the catheter over the guidewire 5.

The tube 3 tapers distally radially outwardly adjacent to the distal end 9. This tapered part 9 of the tube 3 is fixedly attached to the interior surface of the catheter shaft 2, for example by means of a weld joint 10, and the proximal end of the tube 3 is also fixedly attached to the interior surface of the catheter shaft 2. The inner tube 3 is preferably welded to the outer tube 2 at both the flared section 9 and at the RX exit port 4.

The flare on the distal end 9 of the inner tube 3 provides a weld zone to the interior surface of the outer tube 2 and a guiding face to direct the guidewire 5 through the RX port 4 during back loading of the catheter.

The inner tube 3 and the outer tube 2 are welded to the polymer jacket 7 of the proximal shaft wire 6 along a welding region 8 (FIG. 2).

It will be appreciated that other means of attachment may be employed in addition to or as an alternative to welding.

As illustrated in FIG. 1, the distal end 9 of the tube 3 is spaced proximally from the distal end 1 of the catheter shaft 2 to provide sufficient space in the guidewire lumen of the catheter shaft 2 for retrieval of the embolic protection filter 50 into the guidewire lumen. The stepping back of the inner tube 3 from the distal end 1 of the catheter 2 means that the distal 100 mm tip retains high trackability from the low modulus outer tube 2.

The inner tube 3 acts to reinforce the catheter shaft 2 to minimise the possibility of buckling or kinking of the catheter shaft 2 during retrieval. The inner tube 3 also acts as a guide to guide passage of the guidewire 5 from the guidewire lumen of the catheter shaft 2 out through the rapid exchange guidewire opening 4.

The tapered distal end 9 of the inner tube 3 provides a convenient means of attaching the inner tube 3 to the outer tube 2. The tapered distal end 9 also assists in guiding the guidewire 5 into the inner tube 3 in the means of a funnel.

The use of a different material on the inner tube 3 allows the use of a material with a lower coefficient of friction to reduce wire movement forces.

As illustrated in FIGS. 2(c) and 2(d), the wire 6 may extend distally into the attachment region 10, and the distal end of the wire 6 may be fixedly attached to the inner tube 3 and the catheter shaft 2 at the attachment region 10, for example by means of a weld joint.

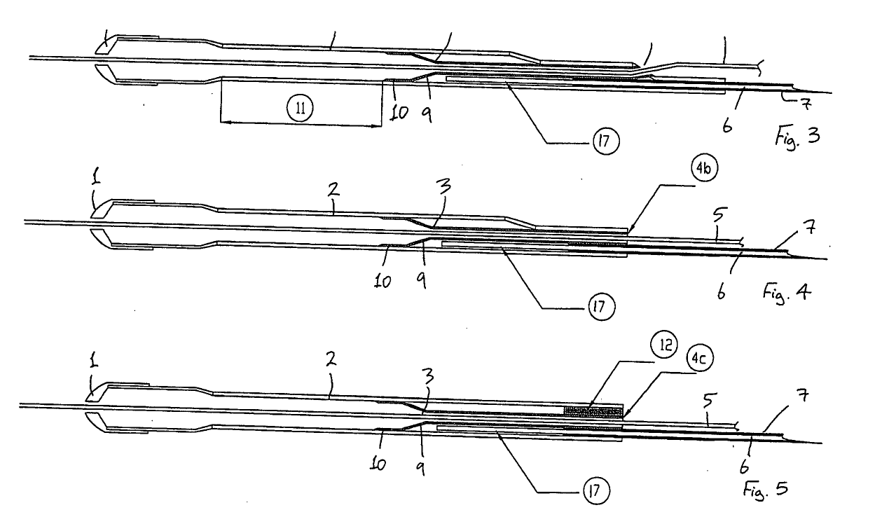

The distance 11 between the distal end 9 of the inner tube 3 and the distal end 1 of the catheter shaft 2 may be varied to vary the degree of reinforcement provided, as illustrated in FIG. 3.

The distal region 17 of the wire 6 may remain untapered, as illustrated in FIG. 3.

In the retrieval catheter of FIG. 4, the guidewire opening 4b faces proximally in a direction substantially parallel to the longitudinal axis of the catheter shaft 2. In this case both the catheter shaft 2 and the inner tube 3 terminate at the guidewire opening 4b to achieve the parallel exit rapid exchange port 4b. The catheter shaft 2 also tapers proximally radially inwardly towards the guidewire opening 4b.

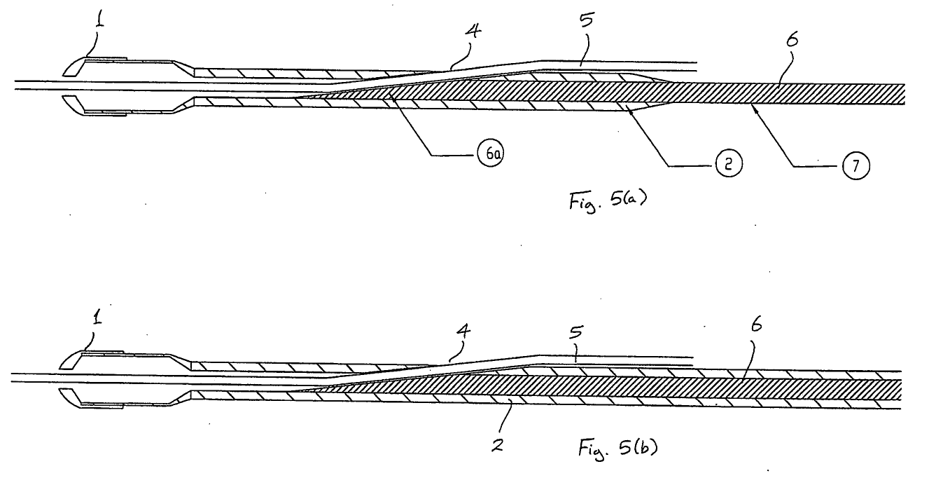

The retrieval catheter of FIG. 5 includes a filler material 12 at the guidewire opening 4c. The catheter shaft 2 does not taper inwardly in this case.

In the retrieval catheter of FIG. 5(a), no inner tube is provided. Instead the wire 6 has a tapered or skived distal end face 6a. The tapered distal end 6a acts as a guide to guide passage of the guidewire 5 out through the guidewire opening 4.

The outer shaft 2 may terminate just proximally of the guidewire opening 4 (FIG. 5(a)). Alternatively the outer shaft 2 may extend proximally over the wire 6 (FIG. 5(b).

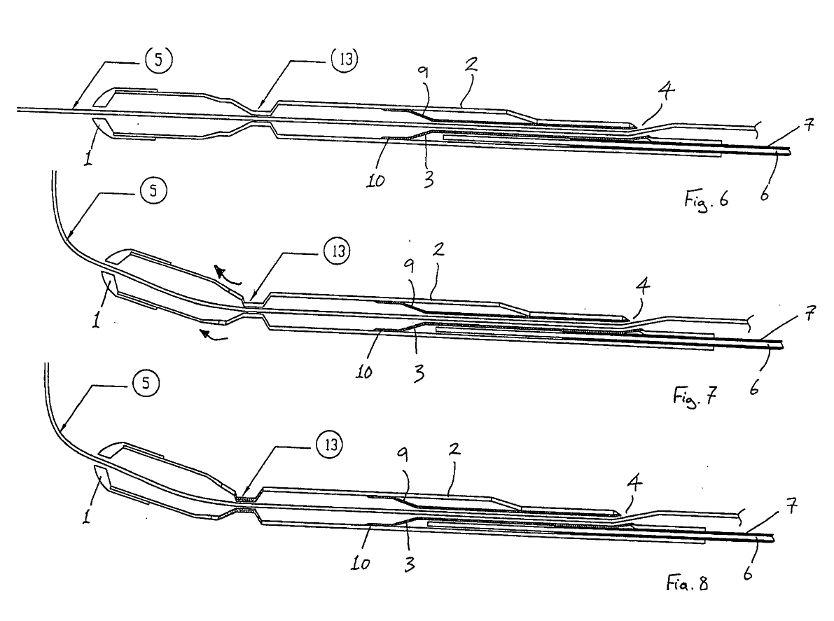

The retrieval catheter of FIG. 6 has a catheter shaft 2 with a hinge region 13. The hinge region 13 is located between the distal end 9 of the inner tube 3 and the distal tip 1, and has a reduced diameter. The hinge region 13 improves distal trackability, as illustrated in FIG. 7.

The hinge region 13 may be of a different material to the remainder of the catheter shaft 2, for example of a more compliant material (FIG. 8).

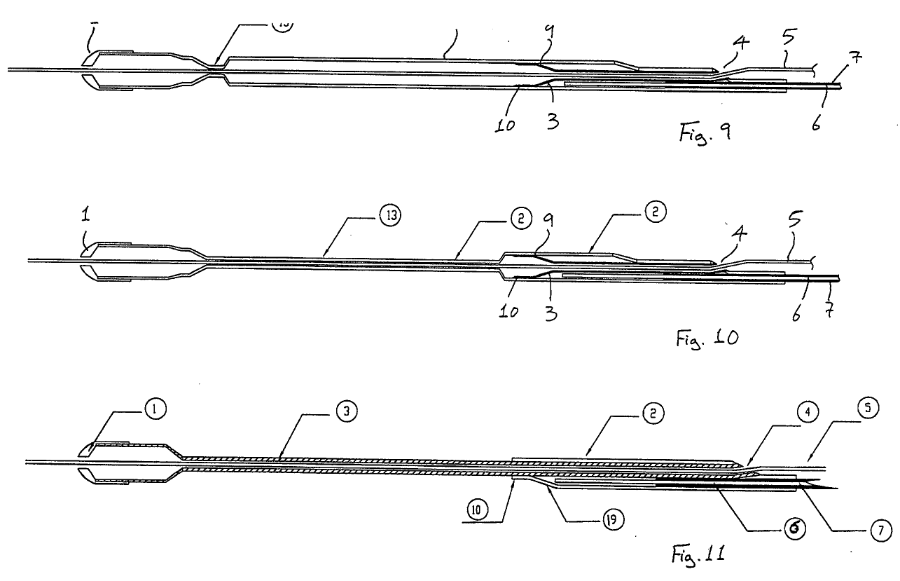

In the retrieval catheter of FIG. 9, a portion 13 of the catheter shaft 2 has a reduced diameter. This reduced diameter portion 13 is located distally of the distal end 9 of the inner tube 3 and proximally of the distal tip 1 (FIG. 9).

By providing this reduced diameter portion 13, the bending/trackability characteristics of the retrieval catheter are enhanced while retaining sufficient compressive strength/pushability. This aspect of the invention is explored in further detail as follows. Reducing the diameter of a shaft has a greater impact on the bending stiffness of the shaft compared to its impact on compression resistance. This is because the shaft bending stiffness is related to the EI value where I has a quadratic relationship to the inner and outer radius of the shaft. The compression resistance is related to EA where A is the cross sectional area, which is related to the square of the inner and outer radius. Therefore, by reducing the shaft diameter it is possible to reduce bending stiffness, but maintain compression resistance by increasing the modulus of the shaft material to maintain the overall EA product.

The distal tip 1 retains the larger diameter to facilitate reception of the embolic protection filter 50 into the tip 1. Also the catheter shaft 2 in the region of the guidewire opening 4 retains the larger diameter to facilitate passage of the guidewire 5 through the guidewire opening 4.

In the case of the retrieval catheter of FIG. 9, the reduced diameter portion 13 extends longitudinally along the catheter shaft 2 for a relatively small distance.

It will be appreciated that the reduced diameter portion 13 may alternatively extend longitudinally along the catheter shaft 2 for a relatively large distance. For example in the retrieval catheter of FIG. 10, the reduced diameter portion 13 extends for substantially the full distance from the distal end 9 of the inner tube 3 to the proximal end of the distal tip 1.

In the retrieval catheter of FIGS. 11 and 12, the inner tube 3 extends longitudinally to the distal tip 1, and the catheter shaft 2 terminates just distally of the distal end of the wire 6. The catheter shaft 2 tapers distally inwardly at a taper region 19 to approach the outer diameter of the inner tube 3, and is fixedly attached to the inner tube 3 at an attachment region 10, for example by means of a weld joint.

The retrieval catheter of FIGS. 11 and 12 also enjoys enhanced bending/trackability characteristics while retaining sufficient compressive strength/pushability.

In further detail, the catheter shaft 2 provides reinforcement to the inner tube 3, in particular in the region of the guidewire opening 4. As illustrated in FIGS. 11 and 12, the inner tube 3 extends distally to the distal tip 1. The catheter shaft 2 and the inner tube 3 are attached to the polymer jacket 7 of the inner wire 6 along the attachment region 8 (FIG. 12). One possible means of attachment is by means of welding.

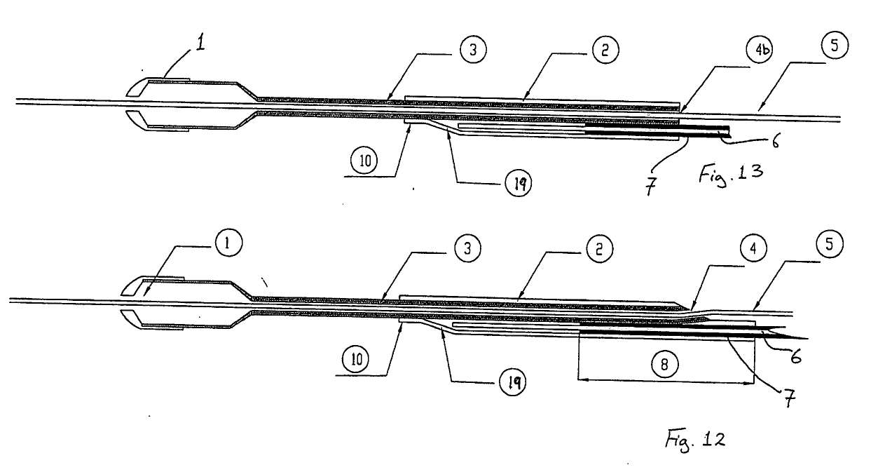

The retrieval catheter of FIG. 13 is similar to the retrieval catheter of FIGS. 11 and 12. In this case, the guidewire opening 4b faces proximally in a direction substantially parallel to the longitudinal axis of the catheter shaft 2. Both the catheter shaft 2 and the inner tube 3 terminate at the guidewire opening 4b to achieve the parallel exit rapid exchange port 4b.

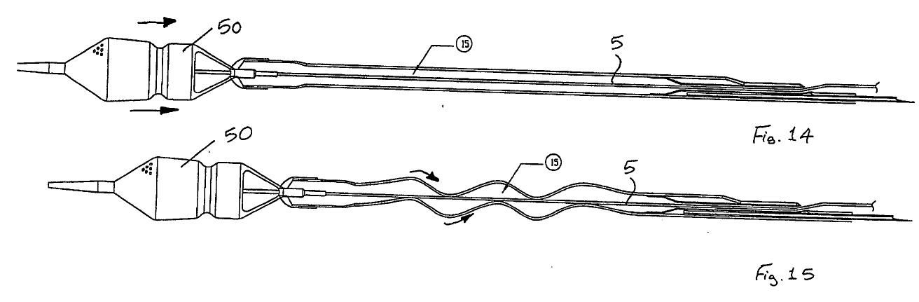

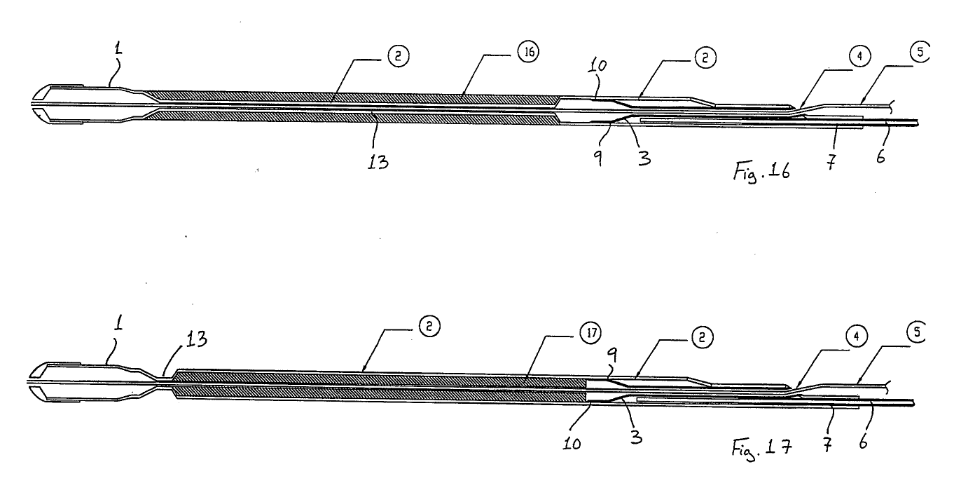

A further advantage of having a reduced diameter portion with the retrieval catheter of the invention is illustrated with respect to FIGS. 14 and 15. The reduced diameter portion 13, such as those illustrated in FIGS. 6 to 13, assists in keeping the retrieval catheter centred on the guidewire 5. In particular because the guidewire 5 is under tension, this arrangement results in the guidewire 5 acting as a support to minimise the possibility of buckling or snaking of the catheter shaft 2 during retrieval of the embolic protection filter 50.

FIGS. 14 and 15 illustrate the possibility of the catheter shaft buckling/snaking during retrieval of the embolic protection filter 50. In this case the space within the guidewire lumen 15 may reduce the pushability of the catheter shaft, and may result in buckling/snaking when it is attempted to retrieve the embolic protection filter 50.

FIG. 16 illustrates another retrieval catheter which is similar to the retrieval catheter of FIG. 10. A bulking member 16 is provided radially outwardly of the catheter shaft 2, extending circumferentially around and longitudinally along the reduced diameter portion 13. The bulking member 16 has a diameter substantially equal to the diameter of the catheter shaft 2 and the diameter of the distal tip 1, which results in the outer diameter of the retrieval catheter being substantially constant from the catheter shaft 2 across the bulking member 16 to the distal tip 1. In this manner the retrieval catheter may be passed through a sealing device, such as a Tuohy Borst, with relative ease and without undue blood loss. The bulking member 16 may be provided by any suitable compliant material.

The retrieval catheter of FIG. 17 is similar to the retrieval catheter of FIG. 9. In this case, the bulking member 17 is provided radially inwardly of the catheter shaft 2, distally of the distal end 9 of the inner tube 3 and proximally of the reduced diameter portion 13. The bulking member 17 assists in preventing bulking/snaking of the catheter shaft 2 during retrieval of the embolic protection filter 50.

It will be appreciated that the first reinforcement element 6 may be manufactured using a variety of different materials. For example the element 6 may be a wire, or a hard durometer polymer. Any material which achieves the desired properties of pushability may be employed.

The jacket 7 may be attached to the reinforcement element 6 in a variety of different ways. For example welding, or extruding may be employed.

The use of a small diameter inner tube 3 means that the distal shaft is more constrained around the tensioned guidewire 5 during retrieval of an embolic load. It is thus less likely to snake or concertina under compression load.

The smaller inner tube 3 helps to prevent a kink in the proximal section of the outer tube 2 inhibiting wire movements through the catheter.

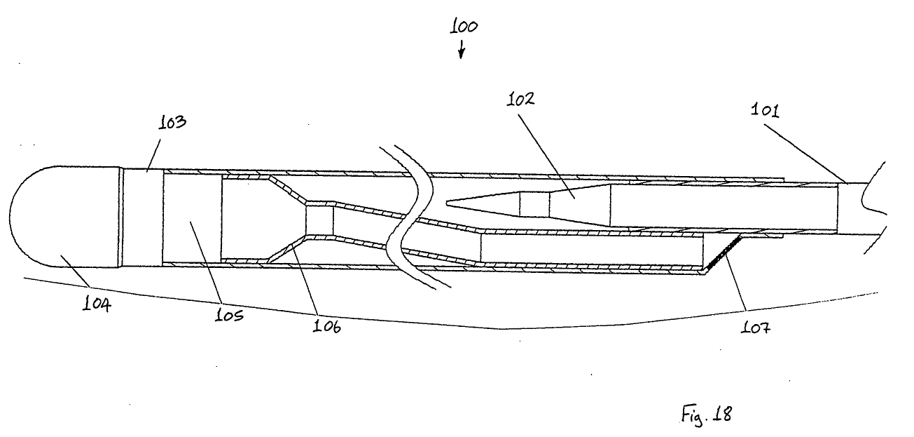

In FIG. 18 there is illustrated another retrieval catheter 100 according to the invention. FIG. 18 illustrates the proximal catheter shaft 101 and the wire support 102 which tapers distally. The distal catheter shaft 103 forms splines for the expansile tip 104. FIG. 18 also illustrates the reception space 105 and the distal flared end 106 of the RX tunnel which acts as a filter abutment surface. The RX port 107 is cut into the sidewall of the distal catheter shaft 103.

By profiling the inner tube geometry, the catheter can achieve zones of varying stiffness without changes in outer profile.

The filter catheters described previously with reference to the drawings may be employed as filter retrieval catheters. In alternative embodiments, the filter catheters may be employed as filter delivery catheters.

The invention is not limited to the embodiments hereinbefore described, with reference to the accompanying drawings, which may be varied in construction and detail.

1. A filter catheter comprising:—
a catheter shaft having a lumen extending at least partially therethrough;
the catheter shaft having a rapid exchange guidewire opening in a wall of the catheter shaft, the guidewire opening being located a substantial distance distally of a proximal end of the catheter for rapid exchange of the catheter over a guidewire; and
at least one reinforcement extending along the catheter shaft within the catheter shaft lumen to reinforce the catheter shaft in the region of the guidewire opening;
the reinforcement comprising a tubular element having a lumen extendeing therethrough aligned with the guidewire opening.

2. A catheter as claimed in claim 1 wherein the tubular element extends distally of the guidewire opening.

3. A catheter as claimed in claim 1 wherein a proximal end of the tubular element is located adjacent to the guidewire opening and a distal end of the tubular element is located distally of the guidewire opening.

4. A catheter as claimed in claim 1 wherein the tubular element terminates proximally of a distal end of the catheter.

5. A catheter as claimed in claim 1 wherein a distal end of the tubular element is attached to the catheter shaft.

6. A catheter as claimed in claim 5 wherein the distal end of the tubular element is attached to an internal surface of the catheter shaft.

7. A catheter as claimed in claim 1 wherein at least part of the tubular element tapers distally radially outwardly.

8. A catheter as claimed in claim 1 wherein the longitudinal axis of the tubular element is radially aligned with the longitudinal axis of the guidewire opening.

9. A catheter as claimed in claim 1 wherein the catheter shaft lumen comprises a guidewire lumen through which a guidewire may be passed for rapid exchange of the catheter over the guidewire.

10. A catheter as claimed in claim 1 wherein the tubular element lumen comprises a guidewire lumen through which a guidewire may be passed for rapid exchange of the catheter over the guidewire.

11. A filter catheter comprising:—
a catheter shaft;
the catheter shaft having a rapid exchange guidewire opening in a wall of the catheter shaft, the guidewire opening being located a substantial distance distally of a proximal end of the catheter for rapid exchange of the catheter over a guidewire; and
at least one reinforcement extending along the catheter shaft to reinforce the catheter shaft in the region of the guidewire opening.

12. A catheter as claimed in claim 11 wherein the catheter shaft has a reception space into which an object may be retrieved.

13. A catheter as claimed in claim 11 wherein the reinforcement is substantially elongate.

14. A catheter as claimed in claim 11 wherein the catheter comprises a first reinforcement and a second reinforcement.

15. A catheter as claimed in claim 11 wherein the reinforcement extends distally of the guidewire opening.

16. A catheter as claimed in claim 15 wherein a proximal end of the reinforcement is located proximally of the guidewire opening and a distal end of the reinforcement is located distally of the guidewire opening.

17. A catheter as claimed in claim 15 wherein a proximal end of the reinforcement is located distally of the guidewire opening and a distal end of the reinforcement is located further distally of the guidewire opening.

18. A catheter as claimed in claim 15 wherein the reinforcement extends distally to a distal end of the catheter.

19. A catheter as claimed in claim 15 wherein the reinforcement terminates proximally of a distal end of the catheter.

20. A catheter as claimed in claim 11 wherein the catheter shaft has a lumen extending at least partially therethrough, and the reinforcement extends along the catheter shaft within the catheter shaft lumen.

21. A catheter as claimed in claim 20 wherein the catheter shaft lumen comprises a guidewire lumen through which a guidewire may be passed for rapid exchange of the catheter over the guidewire.

22. A catheter as claimed in claim 11 wherein the guidewire opening faces in a direction substantially parallel to the longitudinal axis of the catheter shaft.

23. A catheter as claimed in claim 11 wherein the guidewire opening faces in a direction which subtends an acute angle with the longitudinal axis of the catheter shaft.

24. A catheter as claimed in claim 11 wherein the guidewire opening faces proximally.

25. A catheter as claimed in claim 11 wherein the longitudinal axis of the reinforcement is radially offset from the longitudinal axis of the guidewire opening.

26. A catheter as claimed in claim 11 wherein the longitudinal axis of the reinforcement is radially aligned with the longitudinal axis of the guidewire opening.

27. A catheter as claimed in claim 26 wherein the reinforcement has a lumen extending therethrough aligned with the guidewire opening.

28. A catheter as claimed in claim 27 wherein the reinforcement lumen comprises a guidewire lumen through which a guidewire may be passed for rapid exchange of the catheter over the guidewire.

29. A catheter as claimed in claim 28 wherein the reinforcement comprises a guide to guide passage of a guidewire into the reinforcement lumen.

30. A catheter as claimed in claim 29 wherein the guide comprises a funnel.

31. A catheter as claimed in claim 30 wherein the funnel is provided at a distal end of the reinforcement.

32. A catheter as claimed in claim 27 wherein the reinforcement comprises a tubular element.

33. A catheter as claimed in claim 11 wherein the reinforcement has a small cross sectional area relative to the cross sectional area of the guidewire opening.

34. A catheter as claimed in claim 33 wherein the reinforcement comprises a wire element.

35. A catheter as claimed in claim 11 wherein the reinforcement has a cross sectional area substantially equal to the cross sectional area of the guidewire opening.

36. A catheter as claimed in claim 11 wherein at least part of the reinforcement tapers distally radially outwardly.

37. A catheter as claimed in claim 11 wherein at least part of reinforcement tapers distally radially inwardly.

38. A catheter as claimed in claim 36 wherein the tapering part of the reinforcement is located adjacent to a distal end of the reinforcement.

39. A catheter as claimed in claim 11 wherein a distal end of the reinforcement is attached to the catheter shaft.

40. A catheter as claimed in claim 11 wherein a distal end of the reinforcement is detached from the catheter shaft.

41. A catheter as claimed in claim 11 wherein a proximal end of the reinforcement is attached to the catheter shaft.

42. A catheter as claimed in claim 11 wherein the stiffness of the reinforcement is greater than the stiffness of the catheter shaft.

43. A catheter as claimed in claim 11 wherein the second moment of area of the catheter shaft is greater than the second moment of area of the reinforcement.

44. A catheter as claimed in claim 11 wherein the catheter shaft tapers proximally radially inwardly towards the guidewire opening.

45. A catheter as claimed in claim 11 wherein the catheter comprises a guide to guide passage of a guidewire through the guidewire opening.

46. A catheter as claimed in claim 45 wherein the reinforcement comprises the guide.

47. A catheter as claimed in claim 46 wherein the guide is provided by an end face of the reinforcement.

48. A catheter as claimed in claim 11 wherein the catheter shaft has a substantially uniform outer diameter from the guidewire opening to a distal end of the catheter shaft.

49. A catheter as claimed in claim 11 wherein the outer diameter of the catheter shaft varies along the length of the catheter between the guidewire opening and the distal end of the catheter shaft.

50. A catheter as claimed in claim 49 wherein the catheter shaft has a reduced diameter portion.

51. A catheter as claimed in claim 40 wherein the reduced diameter portion extends longitudinally for a part of the distance between the guidewire opening and the distal end of the catheter shaft.

52. A catheter as claimed in claim 40 wherein the reduced diameter portion extends longitudinally for substantially the full distance between the guidewire opening and the distal end of the catheter shaft.

53. A filter retrieval catheter as claimed in claim 1.

54. A filter delivery catheter as claimed in claim 1.
